(19) United States
(12) Patent Application Publication
(10) Pub. No.: US 2010/0130763 A1
(43) Pub. Date: May 27, 2010

(54) PROCESSES FOR THE PRODUCTION OF FATTY ACID ALKYL ESTERS

(75) Inventor: Yong Gao, Carbdondale, IL (US)

Correspondence Address: POLSINELLI SHUGHART PC, 700 W. 47TH STREET, SUITE 1000, KANSAS CITY, MO, 64112-1802, US

(73) Assignee: SOUTHERN ILLINOIS UNIVERSITY CARBONDALE, Carbondale, IL (US)

(21) Appl. No.: 12/517,315

(22) PCT Filed: Dec. 6, 2007
(86) PCT No.: PCT/US07/86573

§ 371 Date: Jan. 26, 2010

Related U.S. Application Data
(60) Provisional application No. 60/868,755, filed on Dec. 6, 2006.

(30) Foreign Application Priority Data
May 23, 2007 (US) 11752666

(51) Int. Cl. C07C 51/00

(52) U.S. Cl. 554/167; 554/124

(57) ABSTRACT

The present invention provides processes for the formation of fatty acid alkyl esters, which may be used as biodiesel. In particular, the invention provides an esterification process in which a lipid material is contacted with an alcohol in the presence of a metal halide or metal alkoxide conjugated to a solid support. Also provided is a transesterification process in which a lipid material is contacted with an alcohol in the presence of an N-heterocyclic carbene. Also provided is a combination esterification and transesterification process in which a lipid material is contacted with a metal halide or metal alkoxide conjugated to a solid support, and then is contacted with an N-heterocyclic carbene or an alkaline catalyst.

### GOVERNMENTAL RIGHTS

The present invention was supported by funding from the National Science Foundation through a Faculty Early Career Development Award (CHE-0343440) and the Small Business Technology Transfer Program (STTR) (IIP-0711652) and funding from the National Institutes of Health (1R15EB007074-01). The United States Government has certain rights in this invention.

### FIELD OF THE INVENTION

The present invention relates to processes for the production of fatty acid esters from lipid materials derived from biological sources.

### BACKGROUND OF THE INVENTION

Conventional energy resources like fossil fuels are becoming limited because of the rapid increase in the worldwide energy demand. This imbalance in energy demand and supply has placed immense pressure not only on consumer prices but also on the environment—prompting extensive research efforts for alternative sustainable energy resources and feedbacks for fuel and chemical production. Biomass is an environmentally friendly renewable resource from which various useful chemicals and fuels can be produced. Biodiesel is the main alternative liquid fuel that is produced from renewable biomass resources, such as vegetable oils.

Biodiesel is a mixture of short chain alkyl (e.g., methyl and ethyl) esters of fatty acids generally derived from the triglycerides of vegetable oils. Although some special diesel engines are able to run on pure vegetable oils, the direct use of vegetable oils in modern diesel engines is generally not satisfactory due to their high viscosity (near 10 times that of petroleum-derived diesel fuels) and other problems such as lower oxidative stability, engine wear, and polymerization of the lubricating oils. Thus, the triglycerides of vegetable oils are generally transesterified to fatty acid alkyl esters, which can be used (alone or blended with conventional diesel fuel) in unmodified diesel engines. The transesterification process used in industrial biodiesel production is usually accomplished by reacting the vegetable oil feedstock with methanol or ethanol in the presence of a homogeneous catalyst. The catalyst may be basic (e.g., NaOH, KOH, NaOMe, or KOMe) or acidic (e.g., H₂SO₄ or HCl).

A major drawback in the production and use of biodiesel is the high cost of the feedstocks. Soybean oil is the major feedstock used in the U.S., whereas sunflower oil and rapeseed oil are widely used in Europe. These feedstocks are not only expensive, but also could be used for human consumption. Thus it is desirable to use alternative cheaper feedstocks such as animal fats, used cooking oils, and industrial waste oils. These cheaper feedstocks, however, have higher concentrations of free fatty acids (FFAs). FFAs are detrimental to transesterification catalysts such as sodium methoxide and potassium hydroxide. As a consequence, complex processes involving extra pretreatment steps to remove FFAs or convert them to esters are usually required for the production of biodiesel from cheaper feedstocks. As a consequence, the increased operation/capital costs of these extra pretreatment steps offset the price advantages of using cheaper feedstocks. In addition, conventional transesterification catalysts such as sodium methoxide are sensitive to water, and thus, extensive steps are usually required to remove water from the triglycerides before the transesterification reaction. These dehydration steps further increase the cost of biodiesel production. Furthermore, the homogenous transesterification catalyst generally remains in the glycerol byproduct after separation of the biodiesel. Since high purity glycerol has greater commercial value than contaminated glycerol, the catalyst has to be removed, further increasing production costs.

Thus, there is a need to identify alternative transesterification processes that use catalysts that are not affected by high levels of FFAs or water, such that cheaper feedstocks could be widely used for biodiesel production. Alternatively, the use of cheaper feedstocks could be facilitated by identifying alternative esterification catalysts that could be readily removed from the reaction products, which could then be directly transesterified. Immobilization of a catalyst on a solid support, for example, would facilitate the ready removal of the catalyst from the reaction products. Furthermore, immobilized catalysts could be recycled and reused, thereby further reducing production costs. Moreover, it economically desirable to use esterification and transesterification catalysts that function efficiently under mild reaction conditions.

### SUMMARY OF THE INVENTION

Among the various aspects of the invention, therefore, is one aspect that provides an esterification process for forming fatty acid alkyl esters. The process comprises contacting a lipid material with an alcohol in the presence of a metal catalyst conjugated to a solid polymer support. The metal catalyst is a halide or an alkoxide of tin, titanium, scandium, or aluminum. The process is conducted at a temperature ranging from about 25° C. to about 75° C., whereby the alcohol reacts with free fatty acids in the lipid material to form fatty acid alkyl esters.

Another aspect of the invention encompasses a transesterification process for forming fatty acid alkyl esters. The process comprises contacting a lipid material with an alcohol in the presence of an N-heterocyclic carbene, wherein alkoxy groups of the glycerides in the lipid material exchange with the hydroxyl groups of the alcohol to form fatty acid alkyl esters.

A further aspect of the invention provides a combination esterification and transesterification process. The process comprises contacting a lipid material with an alcohol in the presence of a halide or an alkoxide of tin, titanium, scandium, or aluminum conjugated to a solid polymer support, whereby the alcohol reacts with free fatty acids (FFAs) in the lipid material to form fatty acid alkyl esters and a FFA-deficient reaction product. The process further comprises contacting the FFA-deficient reaction product with an N-heterocyclic carbene or an alkaline catalyst, whereby the glycerides in the FFA-deficient reaction product are converted to fatty acid alkyl esters.

Other aspects and features of the invention will be in part apparent and in part pointed out hereinafter.

### DESCRIPTION OF THE FIGURES

FIG. 1 depicts an N-heterocyclic carbene covalently bound to a polystyrene solid support.

FIG. 2 depicts an N-heterocyclic carbene bonded to a silica solid support via a linker.

FIG. 3 illustrates the progress of a transesterification reaction catalyzed by a N-heterocyclic carbine. The percent of glyceride conversion is plotted against time in the presence of 1 mol % of 1,3-bis(1-adamantyl)imidazol-2-ylidene.

### DETAILED DESCRIPTION OF THE INVENTION

Advantageously, processes have been discovered for producing fatty acid alkyl esters from, preferably, cheap feedstocks that contain significant amounts of free fatty acids and/or water. First, an esterification process that utilizes a metal halide or metal alkoxide of tin, titanium, scandium, or aluminum conjugated to a solid polymer support has been developed. These metal catalysts function well under mild reaction conditions, and the immobilized catalysts may be readily recovered from the reaction products, recycled, and reused. Second, a transesterification process that utilizes an N-heterocyclic carbene catalyst has been developed. These carbene catalysts also function well under mild reaction conditions, as well as in the presence of significant levels of water. The N-heterocyclic carbene catalysts may also be immobilized by conjugation to a solid support, which permits their recovery, recycling, and reuse. Third, a process that combines both the esterification and transesterification processes of the invention has been developed. Overall, these processes reduce the capitol costs of producing fatty acid esters for biodiesel via the use of cheap feedstuffs, the elimination of pre-treatment steps, and the reuse of catalysts.

### I. Esterification Process

One aspect of the present invention provides a process for forming a fatty acid alkyl ester via an esterification reaction. As used herein, an esterification reaction refers to the chemical process of condensing fatty acids with an alcohol. The process comprises contacting a biological lipid material comprising free fatty acids with an alcohol in the presence of a metal catalyst conjugated to a solid polymer support. The metal catalyst may be an alkoxide or halide derivative of tin, titanium, scandium, or aluminum. In general, the metal halide or the metal alkoxide functions as a catalyst to accelerate the rate of the reaction but is not consumed in the reaction. Conjugation of the metal halide or alkoxide to a solid polymer support permits recovery of the immobilized catalyst from the reaction products, such that the immobilized catalyst may be recycled and reused. Esterification reactions utilizing immobilized metal catalysts are demonstrated in Examples 9 and 10.

(a) Immobilized Metal Halide or Metal Alkoxide Catalysts

The metal catalyst may be a halide or an alkoxide derivative of tin(IV or III), titanium(IV or III), scandium(III), or aluminum(III).

In one embodiment of the invention, the metal catalyst may be a metal halide having Formula (I):

M¹X₄ (I)

wherein, M¹ is selected from the group consisting of tin(IV) and titanium(IV), and X is a halogen atom selected from the group consisting of F, Cl, Br, and I. Non-limiting examples of M¹X₄ compounds include SnF₄, SnCl₄, SnBr₄, SnI₄, TiF₄, TiCl₄, TiBr₄, and TiI₄. In preferred embodiments, M¹X₄ may be SnCl₄ or TiCl₄. A M¹X₄ compound may also be coordinated with another compound, such as tetrahydrofuran (THF), N,N-dimethylformamide (DMF), an amide, or water. For example, a titanium halide may comprise TiCl₄.(THF)₂, TiBr₄.(THF)₂, TiI₄.(THF)₂, TiCl₄.(DMF)₂, or a similar complex.

In another embodiment, the metal catalyst may be a metal halide having Formula (II):

M²X₃ (II)

wherein, M² is selected from the group consisting of tin(III), titanium(III), scandium(III), and aluminum(III), and X is a halogen atom selected from the group consisting of F, Cl, Br, and I. Non-limiting examples of M²X₃ compounds include ScF₃, ScCl₃, ScBr₃, ScI₃, AlF₃, AlCl₃, AlBr₃, and AlI₃.

In a further embodiment, the metal catalyst may be a metal alkoxide having Formula (IV):

M¹(OR)₄ (IV)

wherein, M¹ is selected from the group consisting of tin(IV) and titanium(IV), and R is an acyl group having from 1 carbon atom to about 6 carbon atoms or an alkyl group having from 1 carbon atom to about 6 carbon atoms. Non-limiting examples of suitable M¹(OR)₄ compounds include titanium(IV) methoxide, titanium(IV) ethanoxide, titanium(IV) isopropoxide, titanium(IV) isobutoxide, tin(IV) tetraacetate, and tin(IV) tetrapropionate.

In still another embodiment, the metal catalyst may be a metal alkoxide having Formula (V):

M²(OR)₃ (V)

wherein, M² is selected from the group consisting of tin(III), titanium(III), scandium(III), and aluminum(III), and R is an acyl group having from 1 carbon atom to about 6 carbon atoms or an alkyl group having from 1 carbon atom to about 6 carbon atoms. Non-limiting examples of M²(OR)₃ compounds include scandium(III) methoxide, scandium(III) ethanoxide, titanium(III) isopropoxide, titanium(III) isobutoxide, aluminum(III) tetraacetate, and aluminum(III) tetrapropionate.

The tin, titanium, scandium, or aluminum halide or alkoxide may also be coordinated with another compound, such as tetrahydrofuran (THF), N,N-dimethylformamide (DMF), an amide, or water. The tin, titanium, scandium, or aluminum halide or the tin, titanium, scandium, or aluminum alkoxide may also be complexed with another metal selected from the group consisting of cobalt(II), copper(II), gallium(III), hafnium(IV), iron(III), nickel, zinc(II), and zirconium(IV). Without departing from the scope of the invention, the catalyst may also comprise more than one metal halide or metal alkoxide.

The above-described metal halide or metal alkoxide is immobilized by conjugation to a solid polymer support. As used herein, a solid polymer support refers to any polymeric material that does not dissolve and remains a solid at ambient temperature, and does not react with the lipid material or the alcohol reactants. A polymer comprises repeated structural units (i.e., monomers) that are connected by covalent chemical bonds. The polymer may be a natural polymer, a synthetic polymer, a semi-synthetic polymer, or a synthetic copolymer. Non-limiting examples of polymers include agarose, cellulose, divinylbenzene, methacrylate, methylmethacrylate, methyl cellulose, nitrocellulose, polyacrylic, polyacrylamide, polyacrylonitrile, polyamide, polyether, polyester, polyethylene, polystyrene, polysulfone, polyvinyl chloride, polyvinylidene. Non-limiting examples of suitable copolymers include acrylonitrile-divinylbenzene copolymers, polystyrene-divinylbenzene copolymers (e.g., chloromethylated styrene-divinylbenzene copolymer or sulphonated styrene-divinylbenzene copolymer), methacrylate-divinylbenzene copolymers, and polyvinyl chloride-divinylbenzene copolymers. In a preferred embodiment, the polymer may be a polystyrene-divinylbenzene copolymer, as demonstrated in Examples 9 and 10. The solid polymer support may have a variety of sizes and forms depending upon the embodiment of the invention. For example, the solid support may be beads, microbeads, nanobeads, solid granules, particles, nanoparticles, resins, powders, fibers, nanofibers, nanotubes, gels, sol-gels, areogels, membranes, or a solid surface coated with a solid polymer support.

Depending upon the particular embodiment, the metal halide or alkoxide may be conjugated to the solid polymer support via covalent bonding or non-covalent bonding. Examples of non-covalently bonding include dative bonding, ionic bonding, hydrogen bonding, metallic bonding, and van der Waals bonding. In an exemplary embodiment, the conjugation is via non-covalent bonding. The metal halide or metal alkoxide may be conjugated directly to the solid polymer support. Alternatively, the metal halide or alkoxide may be conjugated to the solid support by at least one linker. Typically, a linker is a molecule having at least two functional groups, such that the linker is disposed between the solid polymer support and the metal catalyst. Typically, one functional group of the linker is attached to the polymer support by a strong covalent bond, and the other functional group of the linker forms an attachment with the metal catalyst by any of the bonding means mentioned above. The composition of the linker, as well as its length, charge, and hydrophobicity, can and will vary depending upon the metal halide or alkoxide, the type of solid polymer support, and the intended uses of the immobilized metal catalyst. For example, a suitable linker may be a linear alkyl chain having from about four carbon atoms to about eight carbon atoms.

Methods for conjugating a metal halide or alkoxide to a solid polymer support are well known to those skilled in the art. By way of a non-limiting example, the metal catalyst and the solid support may be mixed in the presence of at least one solvent at room temperature; the mixture may be brought to reflux; or the mixture may be heated to a temperature between about 100° C. to about 300° C. The weight ratio between the metal halide or alkoxide and the solid polymer support can and will vary depending upon the reactants. For example, the ratio between the metal catalyst and the solid support may range from about 1:1 to about 1:100. The nature of the solvent can and will vary depending upon the reactants. The duration of the reaction can and will vary, depending upon the temperature and the reactants. Typically, the solvent is removed and the metal salt-solid support conjugate is dried before use.

Those skilled in the art will appreciate that the concentration of the immobilized Sn, Ti, Sc and Al catalyst used in the esterification reaction can and will vary, depending upon the source of the lipid material, the temperature of the reaction, and so forth. In one embodiment, the concentration of the catalyst may range from about 0.2% to about 100% by weight of the lipid material. In another embodiment, the concentration of the catalyst may range from about 0.5% to about 65% by weight of the lipid material. In still another embodiment, the concentration of the catalyst may range from about 1% to about 30% by weight of the lipid material.

(b) Lipid Material

The esterification process comprises contacting a lipid material, such as vegetable oils, animal fats, industrial waste products, and spent cooking oil, among others, with the immobilized metal catalyst, whereby free fatty acids in the lipid material are converted to fatty acid esters.

The lipid material may comprise a mixture of free fatty acids (FFA) and glycerides (e.g., mono-, di-, and/or triglycerides). One skilled in the art will appreciate that different lipid materials have different proportions of free fatty acids and glycerides. For example, crude soybean oil may comprise about 1-5% of FFAs, yellow grease may comprise about 15% of FFAs, and brown grease may comprise more than 90% of FFAs. Thus, the lipid material may comprise from about 0.5% to about 99.9% by weight of FFAs. In one embodiment, the lipid material may comprise from about 0.5% to about 10% by weight of FFAs. In another embodiment, the lipid material may comprise from about 10% to about 20% by weight of FFAs. In still another embodiment, the lipid material may comprise from about 20% to about 40% by weight of FFAs. In yet another embodiment, the lipid material may comprise from about 40% to about 60% by weight of FFAs. In an alternate embodiment, the lipid material may comprise from about 60% to about 99.9% by weight of FFAs.

Suitable lipid materials include vegetable oils, animal fats, algae oils, food-based lipid waste, industrial lipid waste, or combinations thereof. Non-limiting examples of suitable vegetable oils include artichoke oil, camelina oil, canola oil, castor oil, coconut oil, copra oil, corn oil, cottonseed oil, flaxseed oil, hemp oil, jatropha oil, jojoba oil, karanj oil, milk brush/pencil bush oil, mustard seed oil, neem oil, olive oil, palm oil, peanut oil, radish oil, rapeseed oil, rice bran oil, rubber seed oil, safflower oil, sesame oil, soybean oil, sunflower oil, and tung oil. Suitable animal fats include, but are not limited to, blubber, chicken fat, cod liver oil, fish oil, ghee, poultry fat, lard, suet, and tallow. Suitable fish oils include anchovy oil, herring oil, lake trout oil, mackerel oil, menhaden oil, pollock oil, salmon oil, and sardine oil. Non-limiting examples of algae oils include those from Aphanizomenon flos-aquae, Bacilliarophy sp., Botryococcus braunii, Chlorophyceae sp., Crypthecodinium cohnii, Dunaliella tertiolecta, Euglena gracilis, Isochrysis galbana, Nannochloropsis salina, Nannochloris sp., Neochloris oleoabundans, Phaeodactylum tricornutum, Pleurochrysis carterae, Prymnesium parvum, Scenedesmus dimorphus, Schizochytrium sp., Spirulina sp., and Tetraselmis chui. Examples of food-based lipid waste include waste vegetable oil (WVO), spent frying oil, yellow grease, which is the reusable grease obtained from restaurant operations, and brown grease, which is the grease collected via the wastewater stream through a passive trap or interceptor. Suitable examples of industrial lipid waste include deodorizer distillates and acid oils (soapstocks) generated as side streams during the production of oil and detergent products. The acid oil may be an acidulated vegetable oil soapstock, such as a soybean soapstock or a corn oil soapstock. Other examples of industrial lipid waste include tall oils, which are byproducts of the pulping of pinewood, and red oils from the candle industry. As will be appreciated by the skilled artisan, the lipid material may be a combination of materials derived from different sources. For example, the lipid material may be a combination of a vegetable oil and an animal fat.

Generally, the lipid material may be contacted with the immobilized metal catalyst without pretreatment. Those skilled in the art will appreciate that the lipid material may need to be pretreated according to the purity of the fatty acid ester desired. For example, the lipid material may be degummed to remove phosphorous or any other solid residue prior to contacting it with the catalyst. Additionally, since some lipid materials, especially the “cheaper” lipid materials, may contain significant levels of water, the lipid material may be dried such that the water content of the lipid material is less than or equal to about 10% by weight of the lipid material. Furthermore, some lipid materials may be both degummed and dried.

(c) Alcohol

In general, any alcohol may be used in the esterification process of the invention. An alcohol comprises any compound having at least one hydroxyl group bound to a carbon atom of an alkyl or a substituted alkyl group. Thus, an alcohol may be linear, cyclic, or branched, and the hydrocarbyl moiety may be saturated or unsaturated. Alcohols suitable for use in this invention will generally have less than about ten carbon atoms. In one embodiment, the alcohol may have from about eight carbon atoms to about ten carbon atoms. In another embodiment, the alcohol may have from about five carbon atoms to about seven carbon atoms. In a preferred embodiment, the alcohol may have from one carbon atom to about four carbon atoms. Suitable alcohols having from one carbon atom to about four carbon atoms include methanol, ethanol, propanol, isopropanol, butanol, and isobutanol. In an exemplary embodiment, the alcohol used in the reaction may be methanol. It should be noted that combinations of alcohols may also be used in the process of the invention.

The concentration of alcohol used in the esterification reaction can and will vary depending upon a variety of factors, including the source of the lipid material. The concentration of alcohol may range from about 1% to about 2000% by weight of the lipid material. In one embodiment, the concentration of alcohol may range from about 1% to about 50% by weight of the lipid material. In another embodiment, the concentration of alcohol may range from about 50% to about 100% by weight of the lipid material. In an alternate embodiment, the concentration of alcohol may range from about 100% to about 500% by weight of the lipid material. In still another embodiment, the concentration of alcohol may range from about 500% to about 1000% by weight of the lipid material. In yet another embodiment, the concentration of alcohol may range from about 1000% to about 1500% by weight of the lipid material. In another alternate embodiment, the concentration of alcohol may range from about 1500% to about 2000% by weight of the lipid material.

Table A lists various combinations of lipid material and alcohol that may be used in the processes of the invention.

TABLE A
Substrate Combinations

| Lipid Material | Alcohol |
|---|---|
| vegetable oil | methanol |
| vegetable oil | ethanol |
| vegetable oil | propanol |
| vegetable oil | isopropanol |
| vegetable oil | butanol |
| vegetable oil | isobutanol |
| vegetable oil | alcohol combination |
| soybean oil | methanol |
| soybean oil | ethanol |
| soybean oil | propanol |
| soybean oil | isopropanol |
| soybean oil | butanol |
| soybean oil | isobutanol |
| soybean oil | alcohol combination |
| rapeseed (canola) oil | methanol |
| rapeseed (canola) oil | ethanol |
| rapeseed (canola) oil | propanol |
| rapeseed (canola) oil | isopropanol |
| rapeseed (canola) oil | butanol |
| rapeseed (canola) oil | isobutanol |
| rapeseed (canola) oil | alcohol combination |
| animal fat | methanol |
| animal fat | ethanol |
| animal fat | propanol |
| animal fat | isopropanol |
| animal fat | butanol |
| animal fat | isobutanol |
| animal fat | alcohol combination |
| tallow | methanol |
| tallow | ethanol |
| tallow | propanol |
| tallow | isopropanol |
| tallow | butanol |
| tallow | isobutanol |
| tallow | alcohol combination |
| lard | methanol |
| lard | ethanol |
| lard | propanol |
| lard | isopropanol |
| lard | butanol |
| lard | isobutanol |
| lard | alcohol combination |
| chicken fat | methanol |
| chicken fat | ethanol |
| chicken fat | propanol |
| chicken fat | isopropanol |
| chicken fat | butanol |
| chicken fat | isobutanol |
| chicken fat | alcohol combination |
| fish oil | methanol |
| fish oil | ethanol |
| fish oil | propanol |
| fish oil | isopropanol |
| fish oil | butanol |
| fish oil | isobutanol |
| fish oil | alcohol combination |
| vegetable oil and animal fat/oil | methanol |
| vegetable oil and animal fat/oil | ethanol |
| vegetable oil and animal fat/oil | propanol |
| vegetable oil and animal fat/oil | isopropanol |
| vegetable oil and animal fat/oil | butanol |
| vegetable oil and animal fat/oil | isobutanol |
| vegetable oil and animal fat/oil | alcohol combination |
| algae oil | methanol |
| algae oil | ethanol |
| algae oil | propanol |
| algae oil | isopropanol |
| algae oil | butanol |
| algae oil | isobutanol |
| algae oil | alcohol combination |
| waste vegetable oil (WVU) | methanol |
| waste vegetable oil (WVU) | ethanol |
| waste vegetable oil (WVU) | propanol |
| waste vegetable oil (WVU) | isopropanol |
| waste vegetable oil (WVU) | butanol |
| waste vegetable oil (WVU) | isobutanol |
| waste vegetable oil (WVU) | alcohol combination |
| yellow grease | methanol |
| yellow grease | ethanol |
| yellow grease | propanol |
| yellow grease | isopropanol |
| yellow grease | butanol |
| yellow grease | isobutanol |
| yellow grease | alcohol combination |
| acid oil (soapstock) | methanol |
| acid oil (soapstock) | ethanol |
| acid oil (soapstock) | propanol |
| acid oil (soapstock) | isopropanol |
| acid oil (soapstock) | butanol |
| acid oil (soapstock) | isobutanol |
| acid oil (soapstock) | alcohol combination |

### (d) Esterification Reaction Conditions

The temperature at which the esterification reaction of the invention is conducted may vary. In general, the temperature will be below the flash points of the substrates. In general, the temperature of the reaction may range from about 25° C. to about 75° C., and more preferably from about 40° C. to about 70° C. In one embodiment, the temperature of the reaction may be about 45° C. In another embodiment, the temperature of the reaction may be about 50° C. In still another embodiment, the temperature of the reaction may be about 55° C. In yet another embodiment, the temperature of the reaction may be about 60° C. In a preferred embodiment, the temperature of the reaction may be about 65° C.

The pressure under which the reaction is conducted may vary. The pressure may range from low pressures, such as 40-60 kPa (˜6-9 psia) to high pressures, such as 350-1200 kPa (˜50-175 psia). Typically, however, the reaction will be conducted at atmospheric pressure, which is about 100 kPa (˜14.5 psia). The esterification process of the invention may also be conducted in the presence of ultrasound and/or microwave.

The duration of the esterification reaction of the invention can and will vary, depending upon the reaction parameters. Typically, the duration of the reaction will be long enough for the reaction to go to completion, i.e., substantially all of the free fatty acids have been converted into fatty acid esters. Techniques well known in the art, such as gas chromatography (GC), nuclear magnetic resonance (NMR), or mass spectrometry (MS), may be used to determine the completeness of the reaction. The duration of the reaction may range from about five seconds to about 48 hours. In one embodiment, the duration of the reaction may range from about five seconds to about 60 minutes. In another embodiment, the duration of the reaction may range from about one hour to about four hours. In an alternate embodiment, the duration of the reaction may range from about four hours to about eight hours. In yet another embodiment, the duration of the reaction may range from about eight hours to about 12 hours. In another alternate embodiment, the duration of the reaction may range from about 12 hours to about 24 hours. In still another embodiment, the duration of the reaction may range from about 24 hours to about 48 hours. In a preferred embodiment, the duration of the reaction may be about 12 hours.

Typically, the reaction may be performed without an additional organic solvent; that is, in addition to the alcohol substrate described above in section (I)(c).

The esterification process of the invention may be conducted in a batch, a semi-continuous, or a continuous mode. The operations may be suitably carried out using a variety of apparatuses and processing techniques well known to those skilled in the art. Furthermore, some of the operations may be omitted or combined with other operations without departing from the scope of the present invention. In a preferred embodiment, the reaction may be performed in a continuous mode of operation. Accordingly, the immobilized metal salt catalyst may be packed in a catalyst bed for repeated uses in, for example, a continuous stirred tank reactor or in a plug-flow tubular reactor.

(e) Esterification Reaction Products

Upon completion of the esterification reaction, the reaction product solution typically comprises fatty acid alkyl esters, water, glycerides, and optionally, unreacted alcohol. Depending upon the alcohol or alcohols used in the reaction, the fatty acid alkyl esters may be fatty acid methyl esters, fatty acid ethyl esters, fatty acid propyl esters, fatty acid isopropyl esters, fatty acid butyl esters, fatty acid isobutyl esters, or combinations thereof. In a preferred embodiment, the fatty acid alkyl esters may be fatty acid methyl esters.

The yield of fatty acid alkyl esters, under optimal reaction conditions, is typically at least about 90%. Depending upon the reaction conditions and other factors, the yield of fatty acid alkyl esters may be at least about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 97%, about 99%, or about 99.5%.

The reaction products may be subjected to at least one additional chemical reaction to convert the glycerides in the solution to fatty acid alkyl esters, as described below in section (III). Alternatively, the reaction product solution may be post-treated to remove reaction byproducts and/or impurities. For example, the water in the solution may be removed. Alternatively, the alcohol in the solution may be removed. Distillation and other suitable techniques are well known to those skilled in the art.

The immobilized metal halide or metal alkoxide catalyst may be recovered, regenerated, and reused. The method used to recover the immobilized catalyst can and will vary, depending mainly upon the mode of operation of the reaction. In a batch reaction, for example, the immobilized catalyst may be recovered by filtration or centrifugation. In continuous mode operations, however, the immobilized catalyst would typically be retained in a fixed-bed column reactor. The immobilized catalyst may be regenerated for repeated use. For example, the immobilized metal catalyst may be treated with the same metal salt, washed with a solvent, and optionally, dried. Alternatively, the immobilized catalyst may be washed with a solvent and, optionally, dried. The number of times the immobilized catalyst may be reused can and will vary. In general, the yield or efficiency of the reaction decreases with each repeated use. The immobilized catalyst may be used until it is completely spent, i.e., when the percent conversion of free fatty acids into fatty acid esters is less than about 20%, less than about 10%, or less than about 5%. Typically, however, economic considerations generally will dictate that the immobilized catalyst be replaced prior to becoming completely spent. For a particular system, those skilled in the art will be readily able to determine when repeated use of the immobilized catalyst is no longer economically feasible in view of increased fatty acid ester losses and the capital expenditure necessary for replacement of the catalyst.

### II. Transesterification Process

Another aspect of the present invention provides a process for forming a fatty acid alkyl ester via a transesterification reaction in the presence of an N-heterocyclic carbene catalyst. As used herein, a transesterification reaction refers to the chemical process of exchanging the alkoxy group of an ester compound with a hydroxyl group. The process of the invention comprises contacting a lipid material and an alcohol in the presence of an N-heterocyclic carbene catalyst, whereby the glycerides in the lipid material react with the alcohol to form fatty acid alkyl esters and glycerol. The N-heterocyclic carbene may be in solution (i.e., homogenous), as demonstrated in Examples 2-4, or it may be conjugated to a solid support (i.e., heterogeneous), as shown in Examples 5 and 6.

(a) N-heterocyclic Carbene Catalysts

The transesterification reaction may be catalyzed by an N-heterocyclic carbene or its precursor. N-Heterocyclic carbenes suitable for use in the invention generally have Formula (VI), Formula (VII), or Formula (VIII):

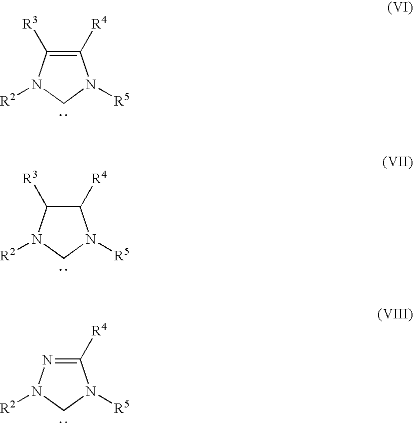

wherein, R² and R⁵ are independently selected from the group consisting of a hydrocarbyl group having from one carbon atom to about 12 carbon atoms and a substituted hydrocarbyl group having from one carbon atom to about 12 carbon atoms; and R³ and R⁴ are independently selected from the group consisting of a hydrogen atom, a halogen atom, and a hydrocarbyl group having from one 1carbon atom to about six carbon atoms.

N-Heterocyclic carbenes suitable for use in the present invention are detailed in Table B.

TABLE B
N-Heterocyclic carbene compounds.

| Compound | | |
|---|---|---|
| Number | Formula Type | Chemical Structure |
| 1 | A compound having Formula (VI) | 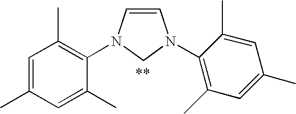 |
| 2 | A compound having Formula (VI) | 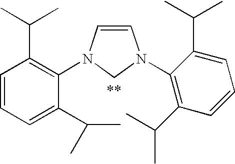 |
| 3 | A compound having Formula (VI) | 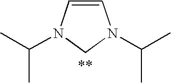 |
| 4 | A compound having Formula (VI) | 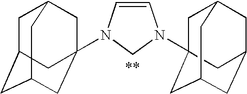 |
| 5 | A compound having Formula (VI) | 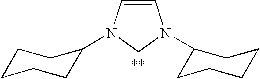 |
| 6 | A compound having Formula (VI) | 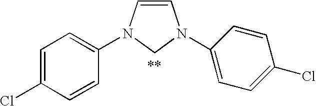 |
| 7 | A compound having Formula (VI) | 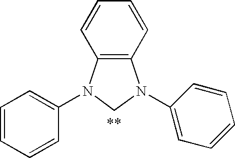 |
| 8 | A compound having Formula (VI) | 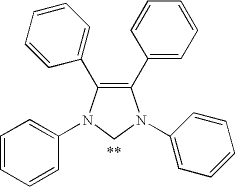 |
| 9 | A compound having Formula (VI) | 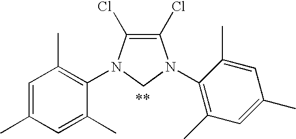 |
| 10 | A compound having Formula (VI) | 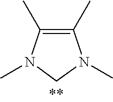 |
| 11 | A compound having Formula (VI) | 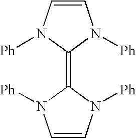 |
| 12 | A compound having Formula (VI) | 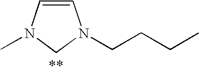 |
| 13 | A compound having Formula (VII) | 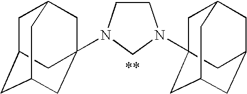 |
| 14 | A compound having Formula (VII) | 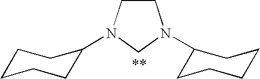 |
| 15 | A compound having Formula (VII) | 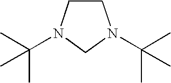 |
| 16 | A compound having Formula (VII) | 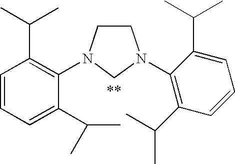 |
| 17 | A compound having Formula (VII) | 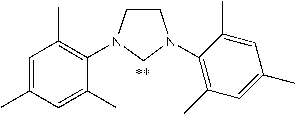 |
| 18 | A compound having Formula (VIII) | 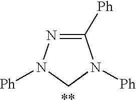 |

In one embodiment, the N-heterocyclic carbene catalyst may be 1,3-bis(2,4,6-trimethylphenyl)imidazol-2-ylidene (IMes, Compound 1 in Table B). In another embodiment, the N-heterocyclic carbene catalyst may be 1,3-bis(2,6-diisopropylphenyl)imidazol-2-ylidene (IprP, Compound 2 in Table B). In still another embodiment, the N-heterocyclic carbene catalyst may be 1,3-diisopropylimidazol-2-ylidene (Ipro, Compound 3 in Table B). In a further embodiment, the N-heterocyclic carbene catalyst may be 1,3-bis(1-adamantyl)imidazol-2-ylidene (IAda, Compound 4 in Table B). In still another embodiment, the N-heterocyclic carbene catalyst may be 1,3-dicyclohexylimidazol-2-ylidene (Icy, Compound 5 in Table B). In another embodiment, the N-heterocyclic carbene catalyst may be 1-butyl-3-methylimidzol-2-ylidene (IBuM, Compound 12 in Table B). In still a further embodiment, the N-heterocyclic carbene catalyst may be 1,3-di-tert-butylimidzolin-2-ylidene (SPro, Compound 15 in Table B). In yet a further embodiment, the N-heterocyclic carbene catalyst may be 1,3,4-triphenyl-1,2,4-triazol-5-ylidene (Compound 18 in Table B). Without limiting the scope of the invention, the N-heterocyclic carbene catalyst may be a metal derivative, a dimer, or a combination of any of the aforementioned compounds.

The N-heterocyclic carbene may be synthesized from a precursor salt of the N-heterocyclic carbene for use in the transesterification reaction, as shown in Example 1. Alternatively, the N-heterocyclic carbene may generated from an alcohol adduct of the N-heterocyclic carbene, as shown in Example 7. Suitable N-heterocyclic carbene alcohol adducts generally have Formula (IX), Formula (X), or Formula (XI):

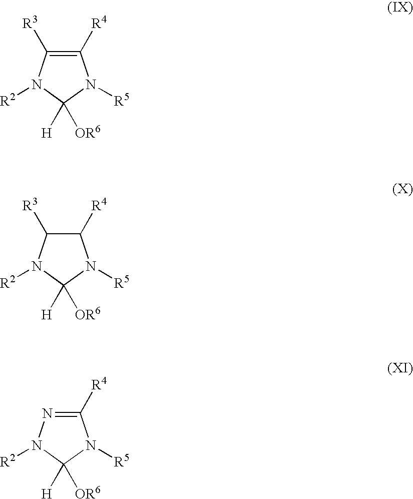

wherein, R² and R⁵ are independently selected from the group consisting of a hydrocarbyl group having from one carbon atom to about 12 carbon atoms and a substituted hydrocarbyl group having from one carbon atom to about 12 carbon atoms; R³ and R⁴ are independently selected from the group consisting of a hydrogen atom, a halogen atom, and a hydrocarbyl group having from one 1carbon atom to about six carbon atoms; and R⁶ is independently selected from the group consisting of a hydrogen atom, and a hydrocarbyl group having from one carbon atom to about six carbon atoms.

Without limiting the scope of the invention, the alcohol adduct of an N-heterocyclic carbene may be generated in situ and used directly to catalyze the transesterification reaction. For example, 1,3-dimesityl-2-methoxyimidazolidine can dissociate and generate a carbene and methanol at room temperature (25° C.), as diagramed below. The in situ-generated carbene may be used to promote the transesterification of glycerides into fatty acid alkyl esters.

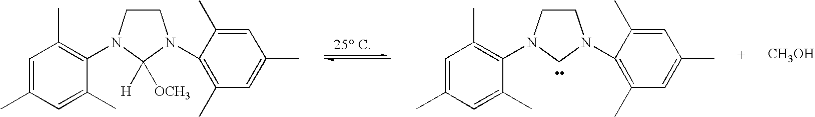

Other alcohol adducts of N-heterocyclic carbenes may be converted into the active carbene at elevated temperature under vacuum. For example, 1,3,4-triphenyl-1,2,4-triazol-5-ylidene may be generated from the corresponding 5-methoxy-1,3,4-triphenyl-4,5-dihydro-1H-1,2,4-triazole by thermal elimination (80 ° C.) of methanol in vacuo (0.1 mbar), as shown below.

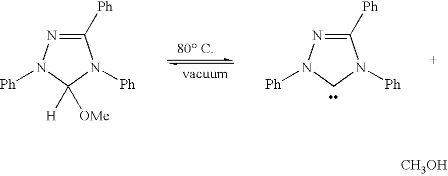

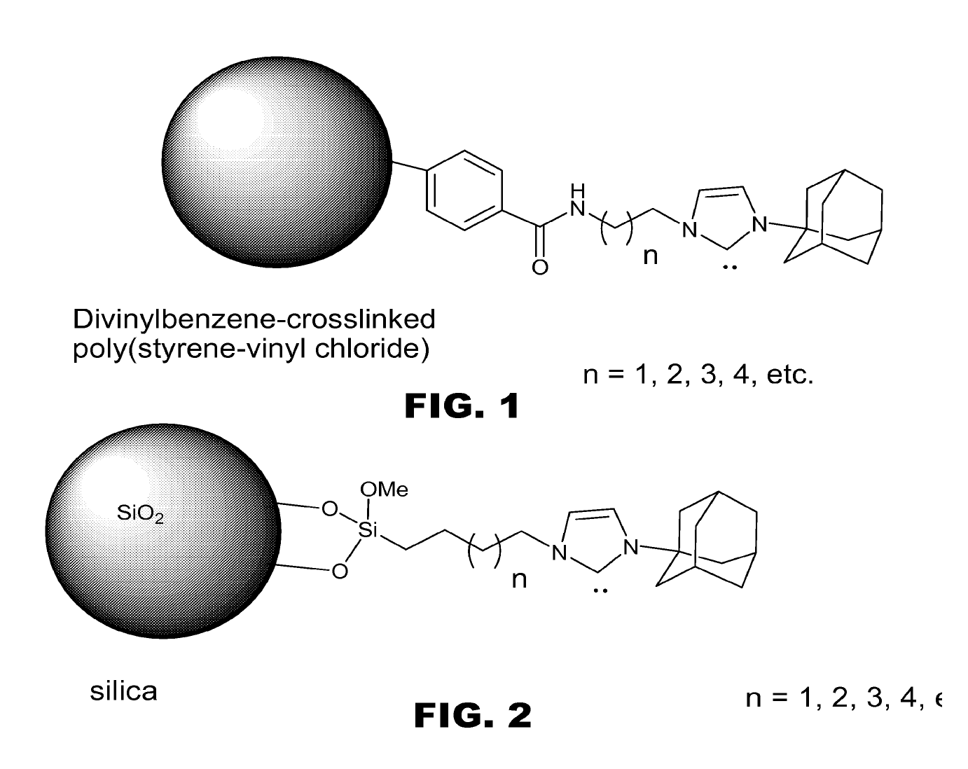

In some embodiments, the N-heterocyclic carbene may be conjugated to a solid support. Suitable solid supports include any of the organic polymers described above in section (I)(a), as well as inorganic solid supports such as silicas (e.g., silicon dioxide, amorphous silica, and microporous or mesoporous silicas, such as SBA-15, MCM-41, FSM-16), alumina, titania, carbondium, zirconia, activated charcoal, zeolites, clays, ceramics, activated carbon, and porous metal supports. In a preferred embodiment, the solid support may be polystyrene-divinylbenzene copolymer beads (see FIG. 1). In another preferred embodiment, the solid support may be a silica resin (see FIG. 2). The N-heterocyclic carbene may be conjugated to the solid support via covalent or non-covalent means; the conjugation may be a direct attachment to the solid support or it may be via a linker, as described above.

The concentration of N-heterocyclic carbene catalyst used in the transesterification reaction can and will vary, depending upon the source of the lipid material, the temperature of the reaction, and so forth. In one embodiment, the concentration of the N-heterocyclic carbene catalyst may range from about 0.02% to about 40% by weight of the starting lipid material. In another embodiment, the concentration of the N-heterocyclic carbene catalyst may range from about 0.5% to about 30% by weight of the starting lipid material. In still another embodiment, the concentration of the N-heterocyclic carbene catalyst may range from about 1% to about 20% by weight of the starting lipid material.

(b) Transesterification Reaction Conditions

The transesterification process of the invention comprises contacting a lipid material with an alcohol in the presence of an N-heterocyclic carbene catalyst, whereby the triglycerides in the lipid material are converted to fatty acid ester. Suitable lipid materials were detailed above in section (I)(b). Suitable alcohols were detailed above in section (I)(c); the concentration of alcohol in the transesterification reaction may range from about 1% to about 2000%, as detailed above.

The temperature at which the transesterification reaction is conducted can and will vary, depending upon the substrates and the catalyst utilized. In general, the temperature of the reaction may range from about 15° C. to about 75° C., or more preferably from about 18° C. to about 40° C. In one embodiment, the temperature of the reaction may be about 30° C. In another embodiment, the temperature of the reaction may be about 25° C. In yet another embodiment, the temperature of the reaction may be about 20° C. In a preferred embodiment, the temperature of the reaction may be at ambient temperature (i.e., about 22° C.).

The pressure under which the transesterification reaction is conducted may vary. The pressure may range from low pressures, such as 40-60 kPa (˜6-9 psia) to high pressures, such as 350-1200 kPa (˜50-175 psia). Typically, however, the reaction will be conducted at atmospheric pressure, which is about 100 kPa (˜14.5 psia). The transesterification process may also be conducted in the presence of ultrasound and/or microwave.

The duration of the transesterification reaction can and will vary, depending upon a variety of factors. Typically, the duration of the reaction will be long enough for the reaction to go to completion, i.e., all of the glycerides have been converted into fatty acid alkyl esters. The progress of the reaction may be monitored by a variety of techniques well known to those of skill in the art. In general, the duration of the reaction may range from about 5 seconds to about 48 hours. In one embodiment, the duration of the reaction may range from about five seconds to about 60 minutes. In another embodiment, the duration of the reaction may range from about one hour to about four hours. In an alternate embodiment, the duration of the reaction may range from about four hours to about eight hours. In yet another embodiment, the duration of the reaction may range from about eight hours to about 12 hours. In another alternate embodiment, the duration of the reaction may range from about 12 hours to about 24 hours. In still another embodiment, the duration of the reaction may range from about 24 hours to about 48 hours. In a preferred embodiment, the duration of the reaction may be about 8 hours.

In general, the transesterification reaction will be conducted without an additional organic solvent; that is, in addition to the alcohol substrate. The reaction may be conducted in a batch, a semi-continuous, or a continuous mode, as detailed above.

(c) Transesterification Reaction Products

Upon completion of the transesterification reaction, the reaction product solution typically comprises fatty acid alkyl esters, glycerol, and optionally, unreacted alcohol. Depending upon the alcohol or alcohols used in the reaction, the fatty acid alkyl esters may be fatty acid methyl esters, fatty acid ethyl esters, fatty acid propyl esters, fatty acid isopropyl esters, fatty acid butyl esters, fatty acid isobutyl esters, or combinations thereof. In a preferred embodiment, the fatty acid alkyl esters may be fatty acid methyl esters.

The yield of fatty acid alkyl esters, under optimal reaction conditions, is typically at least about 90%. Depending upon the reaction conditions and other factors, the yield of fatty acid alkyl esters may be at least about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 97%, about 99%, or about 99.5%.

The solution of fatty alkyl esters may be treated to remove reaction byproducts and/or impurities. In one embodiment, the post treatment process may include distillation to remove the alcohol from the biodiesel solution. In another embodiment, the post treatment process may include vacuum drying to remove water from the biodiesel solution. In yet another embodiment, the post treatment process may also include degumming to remove phosphatides or other solid residues from the biodiesel solution. In still another embodiment, the biodiesel solution may be washed with warm water to remove residual catalyst. In yet an alternate embodiment, the post treatment process may include the purification and fraction of fatty acid esters via distillation or vacuum distillation processes. One skilled in the art will know which process or processes to perform and how to perform them.

In embodiments in which the N-heterocyclic carbene catalyst is conjugated on a solid support, the immobilized catalyst may be recovered, regenerated, and reused. The immobilized N-heterocyclic carbene catalyst may be regenerated by treatment with a base, such as potassium t-butoxide (KOtBu) or sodium hydride (NaH), followed by solvent washing and drying. The immobilized catalyst may be repeatedly used until it is no longer economically feasible, as discussed above in section (I)(e).

The fatty acid alkyl esters formed by the processes of the present invention may be commercially useful for fuel compositions, lubricants, emulsifiers, plasticizers, intermediates for the production of products, such as soaps, detergents, or fragrances, and so forth. In an exemplary embodiment, the fatty acid ester may be used as a fuel composition. The fuel composition may be a biodiesel. Alternatively, the fuel composition may be a blend of a petroleum based diesel fuel and biodiesel.

### III. Combined Esterification/Transesterification Process

A further aspect of the invention encompasses a combined esterification and transesterification method for forming fatty acid alkyl esters. The combined process is particularly useful for the formation of fatty acid alkyl esters from a lipid material that has a high concentration of free fatty acids. The combined method comprises contacting the lipid material with an alcohol in the presence of an immobilized halide or alkoxide derivative of tin, titanium, scandium or aluminum, whereby the free fatty acids in the lipid material are esterified to fatty acid esters. The method further comprises contacting the resultant solution with an N-heterocyclic carbene catalyst or an alkaline catalyst, whereby the glycerides in the lipid material are transesterified to fatty acid esters. Example 11 illustrates the combined use of the esterification and transesterification processes to synthesize fatty acid esters from lipid materials having high levels of free fatty acids.

Suitable lipid materials were described above in section (I)(b). Preferred lipid materials include those with high concentrations of FFAs. In one embodiment, the high free fatty acid-containing lipid material may be selected from the group consisting of tallow, ghee, chicken fat, blubber, cod liver oil, poultry fat, lard, and fish oils, such as menhaden oil, anchovy oil, and mackerel oil, among others. In yet another embodiment, the high free fatty acid-containing lipid material may be selected from the group consisting of spent cooking oil (e.g., used frying oil), yellow grease, brown grease, and algae oils. In a further embodiment, the high free fatty acid-containing lipid material may be coconut oil.

Suitable alcohols were detailed above in section (I)(c). Immobilized metal halide and metal alkoxide catalysts, reaction conditions, and reaction products were detailed above in sections (I)(a), (d), and (e), respectively. In some embodiments, the transesterification catalyst may be an N-heterocyclic carbene, which were detailed above in section (II)(a). In other embodiments, the transesterification catalyst may be an alkaline catalyst. In one embodiment, the alkaline catalyst may be sodium hydroxide. In another embodiment, the alkaline catalyst may be potassium hydroxide. In still another embodiment, the alkaline catalyst may be sodium methoxide. In an alternate embodiment, the alkaline catalyst may be potassium methoxide. The concentration of the alkaline catalyst can and will vary, depending upon the starting lipid material and other factors. In general, the concentration of the alkaline catalyst may range from about 1% to about 25% by weight of the starting lipid material. Transesterification reaction conditions and reaction products were described above in sections (II)(b) and (c), respectively.

### Definitions

To facilitate understanding of the invention, a number of terms, as used herein, are defined below:

The term “acyl” denotes a radical having the general formula RCO⁻, provided after the removal of a hydroxyl group from an organic acid. Examples of acyl radicals include alkanoyl and aroyl radicals. Examples of lower alkanoyl radicals include formyl, acetyl, propionyl, butyryl, isobutyryl, valeryl, isovaleryl, pivaloyl, hexanoyl, and trifluoroacetyl.

As used herein, an “alkoxide” refers to an organic group bonded to a negatively charged oxygen atom (i.e., RO⁻).

The term “alkyl” embraces linear, cyclic, or branched hydrocarbon radicals having one carbon atom to about twenty carbon atoms. More preferred alkyl radicals are “lower alkyl” radicals having one carbon atom to about six carbon atoms. Examples of such radicals include methyl, ethyl, n-propyl, isopropyl, n-butyl, isobutyl, sec-butyl, tert-butyl, pentyl, iso-amyl, hexyl, and the like.

The term “biodiesel,” as used herein, refers to a composition of short chain alkyl esters of fatty acids derived from a biological material.

The term “fatty acid,” as used herein, refers to any of a large group of organic acids made up of molecules containing a carboxyl group (—COON) at one end of a usually unbranched hydrocarbon chain. The hydrocarbon chain may have from about 4 to about 24 carbon atoms, or more specifically, from about 12 to about 22 carbons. The hydrocarbon chain may be saturated or unsaturated.

The term “free fatty acid,” as used herein, refers to the fatty acid product upon breakage of an ester link of a glyceride.

The term “glyceride,” as used herein, refers to esters formed from glycerol and fatty acids. Glycerol has three hydroxyl functional groups that may be esterified with one, two, or three fatty acids to form a monoglyceride, a diglyceride, or a triglyceride.

As used herein, the terms “hydrocarbon” and “hydrocarbyl” describe organic compounds or radicals consisting exclusively of the elements carbon and hydrogen. These moieties include alkyl, alkenyl, alkynyl, and aryl moieties. These moieties also include alkyl, alkenyl, alkynyl, and aryl moieties substituted with other aliphatic or cyclic hydrocarbon groups, such as alkaryl, alkenaryl and alkynaryl. Unless otherwise indicated, these moieties preferably comprise 1 to 20 carbon atoms.

The term “lipid material,” as used herein, comprises glycerides and free fatty acids.

The “substituted hydrocarbyl” moieties described herein are hydrocarbyl moieties that are substituted with at least one atom other than carbon, including moieties in which a carbon chain atom is substituted with a heteroatom such as nitrogen, oxygen, silicon, phosphorous, boron, sulfur, or a halogen (i.e., fluorine, chlorine, bromine, iodine) atom. These substituents also include carbocycle, aryl, heterocyclo, alkoxy, alkenoxy, alkynoxy, aryloxy, hydroxy, protected hydroxy, keto, acyl, acyloxy, nitro, amino, amido, nitro, cyano, thiol, ketals, acetals, esters, and ethers.

As various changes could be made in the above-described processes without departing from the scope of the invention, it is intended that all matter contained in the above description and the examples presented below, shall be interpreted as illustrative and not in a limiting sense.

### Examples

The following examples illustrate various embodiments of the invention.

### General Procedures

Soy oil was purchased from local grocery stores and used “as is” without further purification; 1,3-bis(1-adamantyl)imidazol-2-ylidene (IAda) was purchased from Strem Chemicals (Newburyport, Mass.); all other chemicals were obtained from Acros Organics (Somerville, N.J.) or Aldrich (Milwaukee, Wis.) and used without further purification. ¹H NMR spectra were obtained on a Varian VXR-300 system with an Oxford wide-bore magnet and the chemical shifts were reported in parts per million (ppm) downfield relative to tetramethylsilane using the residual proton resonance of solvents as the references (¹H NMR): CDCl₃ d 7.27; CD₂Cl₂ d 5.32 and (¹³C NMR): CDCl₃ d 77.2; CD₂Cl₂ d 54.0. CDCl₃ solvent was pre-mixed with a TMS internal standard.

### Example 1

### Synthesis of N-Heterocyclic Carbenes

N-heterocyclic carbenes were synthesized from their corresponding imidazolium or imidazolinium salts (see Table 1). About 0.8 eq. of potassium tert-butoxide (8.9 mg) and 1 eq. of a 1, 3-disubstituted imidazolium salt (Table 1, entries 1-5) or 1, 3-disubstituted imidazolinium salt (Table 1, entry 7) was dissolved in 2 mL of tetrahydrofuran (THF). The reaction was stirred for 1 h under argon protection at ambient temperature. The solvent was then removed in vacuo. The resulting carbene was used without further purification and was characterized by ¹H NMR and mass spectrometry.

TABLE 1
N-heterocyclic Carbenes and Their Precursors.

| Imidazolium or | | Carbene |
|---|---|---|
| Imidazolinium Salt | Carbene Product | Abbreviation |
| 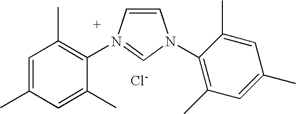 | 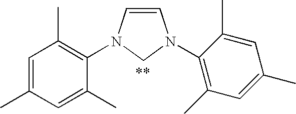 | IMes |
| 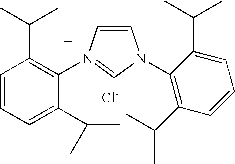 | 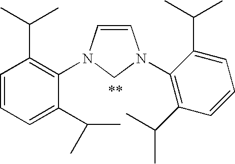 | IprP |
| 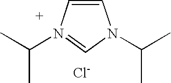 | 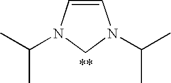 | Ipro |
| 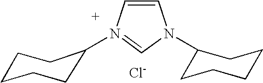 | 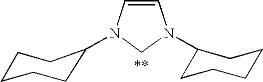 | Icy |
| 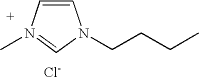 | 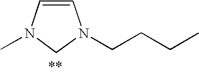 | IbuM |
| | 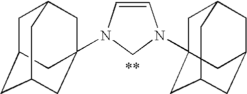 | IAda |
| 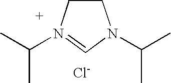 | 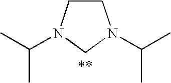 | SPro |

### Example 2

### N-Heterocyclic Carbene-Catalyzed Transesterification Reaction

The general scheme for an N-heterocyclic carbene-catalyzed transesterification reaction is shown below.

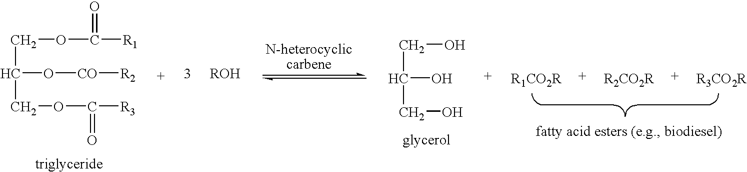

A mixture of soy oil (869 mg) and methanol (100 mg) was treated with a catalytic amount of heterocyclic carbene (1-5 mol %) at ambient temperature (21-23° C.). After 5 min, 200 mL of the reaction mixture was transferred to a small flask containing 20 mL of oleic acid. Methanol was then removed in vacuo and 10 mL of the residue was added to an NMR tube with 600 mL of CDCl₃ for NMR experiments. The reaction yields were determined by ¹H NMR experiments with an internal standard. The conversion yields for the different carbine catalysts are presented in Table 2.

TABLE 2
Reaction Yields (in the absence of water)

| Carbene Catalyst | Yield |
|---|---|
| IAda | >99% |
| IPro | >99% |
| ICy | >99% |
| IBuM | >99% |
| IMes | 17% |
| SPro | 13% |
| IPrP | 12% |

### Example 3

### N-Heterocyclic Carbene-Catalyzed Transesterification Reaction in the Presence of Water

Transesterification reactions were repeated as described above in Example 2, except that the methanol was pre-mixed with 10% water. Table 3 below summarizes the reaction yields under the different carbene catalysts in the presence of a significant amount of water contamination.

TABLE 3
Reaction Yields (in the presence of water)

| Carbene Catalyst | Yield |
|---|---|
| IBuM | >99% |
| IPrP | 93% |
| IMes | 85% |
| ICy | 81% |
| IAda | 65.5% |
| IPro | 62.5% |

### Example 4

### Kinetic Analysis of a Transesterification Reaction

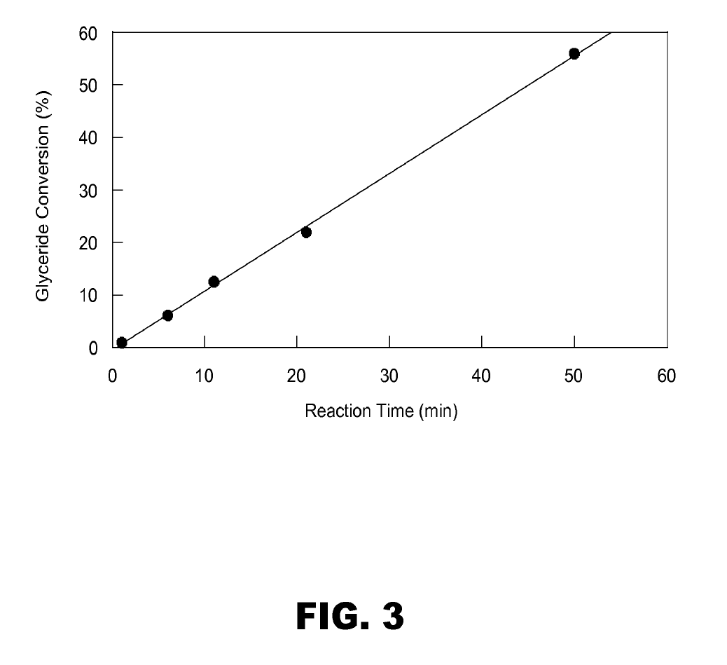

A transesterification reaction was performed in the presence of the catalyst by !Ada as described above in Example 2, except that the amount of catalyst was 1 mol % and that, at fixed time intervals, a 200 mL aliquot was removed from the reaction mixture for NMR analyses. The reaction was linear for at least 50 min (FIG. 3).

### Example 5

### Synthesis and Use of Polymer-Supported N-Heterocyclic Carbenes: Direct Incorporation of the Carbene Precursor into the Polymer Backbone

Immobilized N-heterocyclic carbine catalysts were made by incorporating the carbene directly into the backbone of low crosslinked polymers or high crosslinked polymers. The efficiency of each immobilized N-heterocyclic carbine catalyst was then tested in a transesterification reaction.

Low crosslinked polymer. To a mixture of styrene (5.2 g), divinylbenzene (DVB, 0.26 g), and 1-vinylimidazole (0.94 g) in dimethylformamide (DMF, 6 mL) and heptane (3 mL), was added 2,2′-azobis(2-methylpropionitrile) (AIBN, 0.3 g). The mixture was brought to 100° C. After 16 h, the reaction was cooled down to ambient temperature (21-23° C.). The resultant polymer was filtered and washed with methanol (50 mL×3) and water (50 mL×3) and dried to yield a polymer (3.8 g), with 4% DVB crosslinkage.

The polymer (1.28 g) was suspended in 10 mL of dichloromethane, and methyl iodide (0.3 g) was added. The mixture was stirred for 12 h. Evaporation of the solvent in vacuo gave rise to a methylated polymer (1.27 g). The methylated polymer (0.756 g) was then suspended in dry THF (10 mL) and was treated with KOt-Bu (0.1 g). After 2 h at ambient temperature, the solvent was evaporated in vacuo and the polymer was washed with dry THF and ether to give a polymer-supported carbene catalyst.

The transesterification reaction was carried out at ambient temperature. To a mixture of soybean oil (0.35 g) and methanol (0.75 g) was added the aforementioned polymer-supported carbene catalyst (15 mg). The mixture was stirred at ambient temperature. At a fixed time interval, 200 mL of the sample was taken out of the reaction mixture. ¹H NMR experiments confirmed that after 30 min, 64% of the triglycerides in the soybean oil were converted to the corresponding fatty acid methyl ester. The transesterification reaction was completed within 8 h. The structure of the biodiesel product was determined by ¹H NMR experiments.

High crosslinked polymer. To a mixture of styrene (48 mmol), 1-vinylimidazole (13.7 mmol), and DVB (20.6 mmol) in the mixed solvents of toluene (10 mL) and heptane (10 mL), was added AIBN (2.74 mmol). After 12h at 100° C., the mixture was cooled down to ambient temperature. The solvent was removed in vacuo and the residue was washed with water (50 mL×2) and methanol (50 mL×2) to give 3.6 g of the polymer, with 25% DVB crosslinkage.

The resultant polymer (0.5 g) was treated with CH₃I (0.215 g) in 10 mL of dichloromethane. The suspension was stirred at ambient temperature for 2 h and the methylated polymer was isolated via filtration and dried. The formation of the carbene was carried out using the following procedure: the methylated polymer (0.38 g) in 15 mL of THF was treated with potassium tert-butoxide (KOtBu, 44 mg). After 2 h, the polymer was collected via filtration and washed with dry THF (10 mL×2) to yield the polymer-supported carbene.

The immobilized catalyst was used in a transesterification reaction. For this, a mixture of soybean oil (8.86 g) and methanol (10.42 mL) was added to the highly-crosslinked polymer-supported carbene catalyst (0.317 g). It was found that over 66% of transesterification conversion was achieved after 12 h. The structure of fatty acid methyl esters was confirmed by ¹H NMR.

### Example 6

### Synthesis and Use of Polymer-Supported N-Heterocyclic Carbenes: Functionalization to Introduce the Carbene Precursor to a Pre-Synthesized Polymer

Two different immobilized N-heterocyclic carbine catalysts were made by introducing the carbene precursor to different commercially available polymers. The efficiency of each immobilized N-heterocyclic carbine catalyst was then tested in a transesterification reaction.

A mixture of commercial chloromethylated polystyrene: 1% divinylbenzene copolymer beads (Acros Organics, 1 g) and 1-methylimidazole (144 mg) in 10 mL of CHCl₃ was brought to 50° C. After 5 h, the solution was cooled to ambient temperature. The beads were filtered off and washed with CHCl₃ (50 mL×3) and ethyl acetate (35 mL×3) and dried. About 100 mg of the resultant polymer beads were suspended in 5 mL of dry THF and treated with KOtBu (12.5 mmol). After 30 min, a mixture of soybean oil (0.448 g) and methanol (50 mg) was introduced. NMR experiments were employed to examine the transesterification reaction progress: at 15 min, over 97% of triglycerides were converted into fatty acid methyl esters; after 2 h, the reaction was complete. The polymer catalyst was recovered via filtration. The glycerin and biodiesel layers were separated and the resulted biodiesel was found to meet D6751 standards (2000 Annual Book of ASTM Standards, American Society for Testing and Materials, Philadelphia, 2000; vols. 05.01-05.05.). The recycled catalyst was subjected to two more rounds of the same reactions: the 2nd round: after 15 min, 65% conversion; 2 h, 100% conversion; the 3rd round: after 15 min, 62% conversion; 2 h, >99% conversion.

A mixture of commercial 4-bromobutyl polystyrene beads (NovaBiochem, 0.3 g) and 1-methylimidazole (144 mg) in 10 mL of CHCl₃ was brought to 50° C. After 12 h, the solution was cooled to ambient temperature. The beads were filtered off and washed with CHCl₃ (50 mL×3) and ethyl acetate (35 mL×3) and dried. About 30 mg of the resultant polymer beads were suspended in 5 mL of dry THF and treated with KOtBu (12.5 mmol). After 30 min, a mixture of soybean oil (0.448 g) and methanol (50 mg) was introduced. NMR experiments were employed to examine the transesterification reaction progress: after 2 h, about 25% of triglycerides were converted into biodiesel.

### Example 7

### Synthesis and Use of Alcohol Adduct of N-Heterocyclic Carbene

To a THF solution (10 mL) of 1,3-bis(2,4,6-trimethylphenyl)imidazolinium chloride (0.5 g) was added KOtBu (0.189 g) under argon protection. After 2 h, the solvent was removed and the solid was washed with THF (15 mL×2) to give 1,3-bis(2,4,6-trimethylphenyl)imidazolin-2-ylidene (0.15 g). About 20 mg of 1,3-bis(2,4,6-trimethylphenyl)imidazolin-2-ylidene was then treated with methanol (10 mL) in 5 mL of THF. After 1 h, the mixed solvents were removed in vacuo. Then, a mixture of soybean oil (0.345 g) and methanol (75 mg) was introduced. It was found that at ambient temperature, after 2 h, about 35% of triglycerides were converted into biodiesel.

### Example 8

### Synthesis and Use of N-Heterocyclic Carbene and Metal Complex

The titanium and N-heterocyclic carbene complex (Ti—NHC) was synthesized using the protocol of Patel et al. (Chem. Commun. 2006, 10:1124-1126).

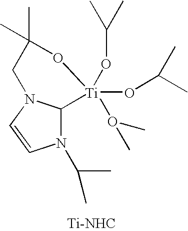

To a stirred solution of soybean oil (0.345 g) and methanol (65 mg) was added Ti—NHC (5 mol %). After 30 min, ¹H NMR analysis confirmed that about 18% of the triglycerides were converted into fatty acid methyl esters. The structure of the product was confirmed by ¹H NMR.

### Example 9

### Synthesis and Use of Polymer-Supported Metal Complexes: Aminol Polymer Metal Complexes

Polymer-supported tin(IV or III), titanium(IV or III), scandium(III) and aluminum(III) halide and alkoxide catalysts were synthesized and tested for promoting esterification reactions of lipid materials.

A representative scheme for the synthesis of an aminol polymer-metal complex is presented below.

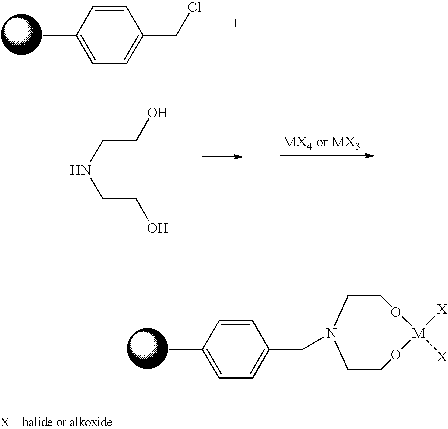

A mixture of diethanolamine (10.5 g) and sodium carbonate (5.8 g) was mixed with 40 mL of DMF in a round bottom flask under argon. The mixture was brought to 80° C. and then chloromethylated polystyrene: 1% divinylbenzene copolymer beads (5 g) were added. After 48 h at 80° C., the mixture was cooled to ambient temperature and the polymer was filtered. The polymer was then washed with water (100 mL×4) and methanol (100 mL×4) and dried to yield 5.5 g of an aminol-loaded polymer.

A metal (Ti, Sn, Zr, Sc, or Al) halide or alkoxide (1.2 mmol) was added to 0.5 g of the aminol-loaded polymer in 10 mL of THF. After 4 h, the polymer was filtered and washed with THF (30 mL×2) and dried to give the polymer-metal complex. Elemental analysis experiments have confirmed the structure of the complex.

The efficiency of each polymer-metal catalyst complex was evaluated in esterification reactions using several different lipid materials as the starting material (i.e., oleic acid in soybean oil, soy soapstock, or brown grease).

A polymer-metal complex (5 mol %, calculated based on the metal loading) was added to a mixture of oleic acid (1 g) in soybean oil (500 mg). The solution was brought to 65° C. After 12 h, the mixture was cooled down to ambient temperature. The polymer was recovered via filtration and excessive methanol was removed. The residue was analyzed by ¹H NMR and titration experiments were performed using AOCS methods (Official Methods and Recommended Practices of the American Oil Chemists' Society, 5th ed.; Firestone, D. Ed.; American Oil Chemists' Society: Champaign, Ill., 1998.) The yields are presented in Table 4.

TABLE 4
Reaction Yield of Oleic Acid
in Soybean Oil.

| Metal salt | Yield (%) |
|---|---|
| ZrCl₄ | 95 |
| Zr(OEt)₄ | 36 |
| TiCl₄ | 100 |
| Ti(OiPr)₄ | 13 |
| SnCl₄ | 100 |
| AlCl₃ | 57 |
| Al(OET)₃ | 11 |
| ScCl₃ | 41 |
| Sc(OiPr)₃ | 7 |
| TiCl₃ | 24 |

Alternatively, an acidulated soybean soapstock (provided by Kappa Products Corporation, Kerry, New Zealand) (184 g, about 70% free fatty acids) was treated with an aminol polymer-metal halide (alkoxide) complex (5 mol %) and methanol (168 g) at 65° C. After 12 h, the solution was cooled to ambient temperature and the polymer was recovered and excessive methanol was removed. The residue was analyzed as described above, and typical yields are presented in Table 5.

TABLE 5
Reaction Yield of Soapstock.

| Metal salt | Yield (%) |
|---|---|
| ZrCl₄ | 99 |
| TiCl₄ | 99 |
| SnCl₄ | 99 |
| AlCl₃ | 30 |

The esterification reaction product formed in the presence of the aminol polymer-TiCl₄ catalyst was further distilled via fractional distillation (10 mm Hg, 5 cm long distillation column). The main fraction was analyzed using AOCS methods and the resultant fatty acid methyl esters were found to meet ASTM D6751 standards. Some key properties are listed in Table 6.

TABLE 6
Properties of Fatty Acid Methyl Esters.

| Biofuel property | Distillate |
|---|---|
| Density @ 15° C. (kg/m³) | 934 |
| Viscosity @ 40° C. (mm²/S) | 4.321 |
| Flashpoint (° C.) | 130.0 |
| Smoke Point (° C.) | 120.8 |
| Water (% mass) | 0.0109 |
| Acid number (mg KOH/g) | 0.27 |
| Free glycerol (% mass) | 0.0126 |
| Total glycerol (% mass) | 0.0612 |

The recovered TiCl₄-polymer complex was subjected to three more rounds of reaction with soybean soapstock, and no significant loss of activity (99%, 12 h) was observed.

The TiCl₄-polymer complex (5 mol %) was added to a mixture of recovered brown grease (restaurant trap grease; sample was pretreated and provided by Commercial waste Management, Inc., Doraville, Ga.) (184 g) and methanol (170 g) at 65° C. After 12 h, the mixture was cooled to ambient temperature and the polymer was recovered via filtration. Analysis of the esterification product showed that the acid number had dropped from 221.63 of the starting material (brown grease) to 0.7 mg KOH/g. ¹H NMR experiments confirmed the formation of fatty acid methyl esters.

### Example 10

### Synthesis and Use of Polymer-Supported Metal Complexes: Phenyl Polymer Metal Complexes

A representative scheme for the synthesis of a phenol polymer-metal complex is presented below.

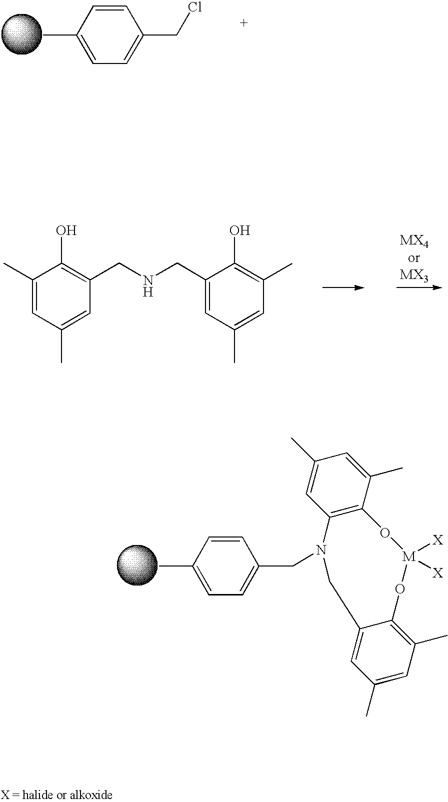

A mixture of chloromethylated polystyrene: 1% divinylbenzene copolymer beads (1.5 g) and 2,2′-methylene-4,4′,6,6′-etramethyldiphenol (synthesized according to Dargaville, et al., Chemistry of Novolac Resins. II, 1996, 1389-1398) (1 g) was mixed with K₂CO₃ (0.48 g) in 20 mL of DMF. After 48 h, the polymer was isolated via filtration and washed with water (50 mL X 3), methanol (30 mL×4), THF (30 mL×3) and DMF (30 mL×2) and dried.

A metal (Ti, Sn, Zr, Sc, or Al) halide or alkoxide (1.2 mmol) was added to 0.5 g of the polymer in 10 mL of THF. After 4 h, the polymer was filtered and washed with THF (30 mL×2) and dried to give the polymer-metal complex. Elemental analysis experiments confirmed the structure of the complex.

For esterification reactions, a polymer-metal complex (5 mol %, calculated based on the metal loading) was added to a mixture of oleic acid (1 g) in soybean oil (500 mg) was added. The solution was brought to 65° C. After 12 h, the mixture was cooled to ambient temperature. The polymer was recovered via filtration and excessive methanol was removed. The residue was analyzed by ¹H NMR and titration experiments using AOCS methods. Typical yields are presented in Table 7.

TABLE 7
Reaction Yields.

| Metal salt | Yield (%) |
|---|---|
| ZrCl₄ | 96 |
| Zr(OEt)₄ | 9 |
| TiCl₄ | 89 |
| Ti(OiPr)₄ | 12 |
| SnCl₄ | 91 |

### Example 11

### Combined Esterification and Transesterification Catalytic Reactions

The following experiments were performed to demonstrate the utility of the above-described reactions for converting high free fatty acid feed stocks into fatty acid esters. The product from the reaction of brown grease and the aminol polymer-TiCl₄ complex (see Example 9) (1 g) was mixed with NaOMe (30 mg) and methanol (1.2 g). The mixture was stirred at ambient temperature for 1 h, and then the reaction mixture was separated into two phases. Excessive methanol was removed in vacuo and the two layers were separated. ¹H NMR experiments and the glycerine analysis (AOCS method Ca14-56) confirmed that over 89% of triglycerides were converted into fatty acid methyl esters.

Alternatively, the product from reaction of acidulated soybean soapstock and the aminol polymer-TiCl₄ complex (see Example 9) was used directly for the next transesterification reaction. The Ti polymer was removed and the filtrate (˜352 g) was treated with the N-heterocyclic carbene, 1-butyl-3-methylimidzol-2-ylidene (0.9 g). (This catalyst was synthesized by dissolving about 0.8 eq. of potassium tert-butoxide and 1 eq. of 1-butyl-3-methylimidazolium salt in 20 mL of dry THF. After 1 h under argon protection, the solvent was removed and the resulting 1-butyl-3-methylimidzol-2-ylidene was added to the esterification reaction mixture.) After 1 h, the transesterification reaction mixture was separated into two phases. Excessive methanol was removed in vacuo, and the two layers were separated. ¹H NMR and glycerine analyses confirmed that the amount of triglycerides dropped from 11% to 0.18%.

1-66. (canceled)

67. A process for forming a fatty acid alkyl ester, the process comprising contacting a solution consisting of two substrates with a metal catalyst conjugated to a solid polymer support, the substrates being a lipid material and an alcohol comprising four carbon atoms or fewer, the metal catalyst being selected from the group consisting of a halide and an alkoxide of a metal selected from the group consisting of aluminum, scandium, tin, titanium, and zirconium, the process occurring at a temperature from about 25° C. to about 75° C., whereby the alcohol reacts with a free fatty acid (FFA) in the lipid material to form the fatty acid alkyl ester and a FFA-deficient reaction product.

68. The process of claim 67, wherein the metal halide is a compound comprising a formula selected from the group consisting of M¹X₄ and M²X₃, and the metal alkoxide is a compound comprising a formula selected from the group consisting of M¹(OR)₄ and M²(OR)₃, wherein:
M¹ is a metal atom selected from the group consisting of tin(IV), titanium(IV), and zirconium(IV);
M² is a metal atom selected from the group consisting of aluminum(III), scandium(III), tin(III), and titanium(III);
R is an acyl group having from one carbon atom to about six carbon atoms or an alkyl group having from one carbon atom to about six carbon atoms; and
X is a halogen atom selected from the group consisting of F, Cl, Br, and I.

69. The process of claim 67, wherein the metal catalyst is coordinated with a compound selected from the group consisting of tetrahydrofuran, N,N-dimethylformamide, an amide, and water.

70. The process of claim 67, wherein the metal catalyst is complexed with a metal selected from the group consisting of cobalt(II), copper(II), gallium(III), hafnium(IV), iron(III), nickel, and zinc(II).

71. The process of claim 67, wherein the solid polymer support is selected from the group consisting of divinylbenzene, methacrylate, polyacrylic, polyacrylamide, polyacrylonitrile, polyamide, polyethylene, polystyrene, polysulfone, polyvinyl chloride, polyvinylidene, polystyrene-divinylbenzene copolymer, methacrylate-divinylbenzene copolymer, and polyvinyl chloride-divinylbenzene copolymer.

72. The process of claim 67, wherein the lipid material is selected from the group consisting of a vegetable oil, an animal fat, an algae oil, a food-based lipid waste, an industrial lipid waste, and a combination thereof, wherein the lipid material comprises from about 0.5% to about 99.9% by weight of FFAs

73. The process of claim 67, wherein the concentration of the alcohol is from about 1% to about 2000% by weight of the lipid material; the concentration of the metal catalyst is from about 0.2% to about 100% by weight of the lipid material;
the process is conducted at a pressure from about 40 kPa to about 1200 kPa and proceeds for no more than about 48 hours; and the conversion of free fatty acids to fatty acid alkyl esters is at least about 90%.

74. The process of claim 67, wherein the lipid material is food-based lipid waste; the alcohol is methanol; the concentration of the alcohol is from about 10% to about 40% by weight of the lipid material; the metal catalyst is tin(IV) tetrachloride; the concentration of the metal catalyst is from about 1% to about 30% by weight of the lipid material; the solid polymer support is a polystyrene-divinylbenzene copolymer; the temperature of the reaction is about 65° C.; the process is conducted at a pressure of about 100 kPa and proceeds for about 12 hours; and the conversion of free fatty acids to fatty acid alkyl esters is at least about 90%.

75. The process of claim 67, further comprising contacting the FFA-deficient reaction product with an N-heterocyclic carbene, whereby a glyceride in the FFA-deficient reaction product is converted to a fatty acid alkyl ester.

76. The process of claim 75, wherein the N-heterocyclic carbene is a compound comprising a formula selected from the group consisting of (VI), (VII), (VIII), (IX), (X), and (XI): 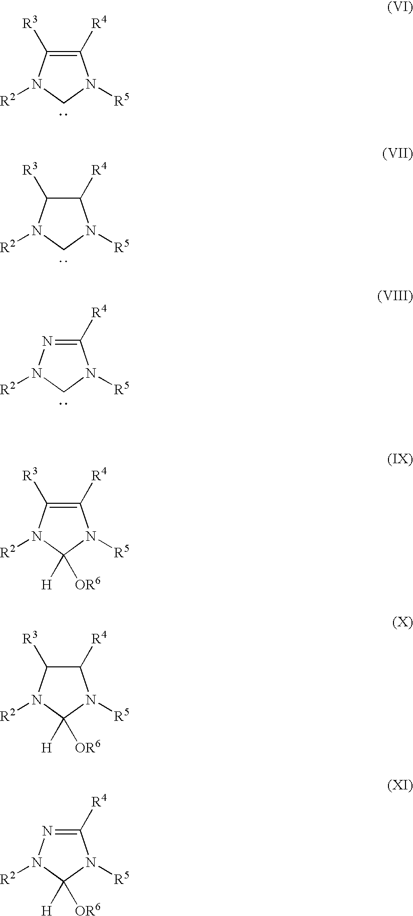
wherein:
R² and R⁵ are independently selected from the group consisting of a hydrocarbyl group having from one carbon atom to about twelve carbon atoms and a substituted hydrocarbyl group having from one carbon atom to about twelve carbon atoms;
R³ and R⁴ are independently selected from the group consisting of a hydrogen atom, a halogen atom, and a hydrocarbyl group having from one carbon atom to about six carbon atoms; and
R⁶ is independently selected from the group consisting of a hydrogen atom, a halogen atom, and a hydrocarbyl group having from one carbon atom to about six carbon atoms.

77. The process of claim 75, wherein the N-heterocyclic carbene is selected from the group consisting of 1,3-bis(2,4,6-trimethylphenyl)imidazol-2-ylidene, 1,3-bis(2,6-diisopropylphenyl)imidazol-2-ylidene, 1,3-diisopropylimidazol-2-ylidene, 1,3-bis(1-adamantyl)imidazol-2-ylidene, 1,3-dicyclohexylimidazol-2-ylidene, 1-butyl-3-methylimidzol-2-ylidene, 1,3-di-tert-butylimidzolin-2-ylidene, 1,3,4-triphenyl-1,2,4-triazol-5-ylidene, a metal derivative, a dimer, and a combination thereof.

78. The process of claim 75, wherein the N-heterocyclic carbene is conjugated to a solid support selected from the group consisting of polymer, copolymer, alumina, silica, and zeolite.

79. The process of claim 75, wherein the concentration of the N-heterocyclic carbene is from about 0.02% to about 40% by weight of the starting lipid material; the process is conducted at temperature from about 15° C. to about 75° C., at a pressure from about 40 kPa to about 1200 kPa, and for no longer than about 48 hours; and the conversion of glycerides to fatty acid alkyl esters is at least about 90%.

80. The process of claim 75, wherein the N-heterocyclic carbene is 1-butyl-3-methylimidzol-2-ylidene; the concentration of the N-heterocyclic carbene is from about 1% to about 20% by weight of the starting lipid material; the process is conducted at temperature of about 22° C., at a pressure of about 100 kPa, and for about 8 hours; and the conversion of glycerides to fatty acid alkyl esters is at least about 90%.

81. A process for forming a fatty acid alkyl ester, the process comprising contacting a lipid material with an alcohol in the presence of an N-heterocyclic carbene, wherein an alkoxy group of a glyceride in the lipid material exchanges with a hydroxyl group of the alcohol to form the fatty acid alkyl ester.

82. The process of claim 81, wherein the N-heterocyclic carbene is a compound comprising a formula selected from the group consisting of (VI), (VII), (VIII), (IX), (X), and (XI): 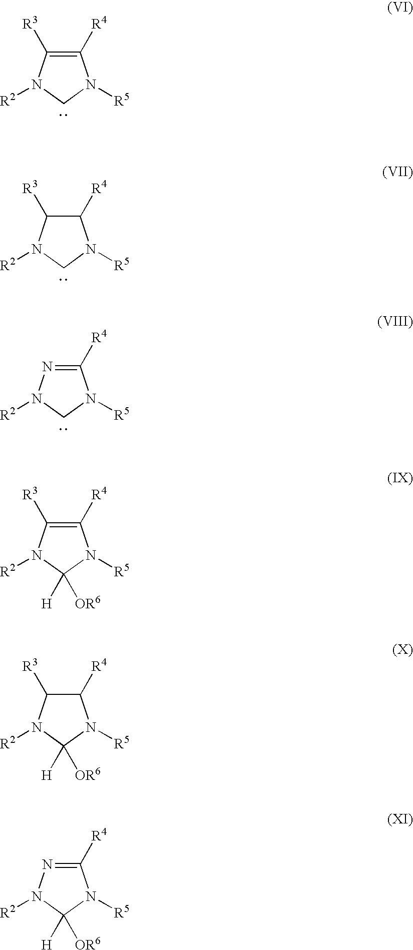
wherein:
R² and R⁵ are independently selected from the group consisting of a hydrocarbyl group having from one carbon atom to about twelve carbon atoms and a substituted hydrocarbyl group having from one carbon atom to about twelve carbon atoms;
R³ and R⁴ are independently selected from the group consisting of a hydrogen atom, a halogen atom, and a hydrocarbyl group having from one carbon atom to about six carbon atoms; and
R⁶ is independently selected from the group consisting of a hydrogen atom, a halogen atom, and a hydrocarbyl group having from one carbon atom to about six carbon atoms.

83. The process of claim 81, wherein the N-heterocyclic carbene is selected from the group consisting of 1,3-bis(2,4,6-trimethylphenyl)imidazol-2-ylidene, 1,3-bis(2,6-diisopropylphenyl)imidazol-2-ylidene, 1,3-diisopropylimidazol-2-ylidene, 1,3-bis(1-adamantyl)imidazol-2-ylidene, 1,3-dicyclohexylimidazol-2-ylidene, 1-butyl-3-methylimidzol-2-ylidene, 1,3-di-tert-butylimidzolin-2-ylidene, 1,3,4-triphenyl-1,2,4-triazol-5-ylidene, a metal derivative, a dimer, and a combination thereof.

84. The process of claim 81, wherein the N-heterocyclic carbene is conjugated to a solid support selected from the group consisting of polymer, copolymer, alumina, silica, and zirconia.

85. The process of claim 81, wherein the lipid material is selected from the group consisting of a vegetable oil, an animal fat, an algae oil, a food-based lipid waste, an industrial lipid waste, and a combination thereof.

86. The process of claim 81, wherein the alcohol is a short chain alcohol selected from the group consisting of methanol, ethanol, propanol, isopropanol, butanol, isobutanol, and a combination thereof.

87. The process of claim 81, wherein the concentration of the alcohol is from about 1% to about 2000% by weight of the lipid material; the concentration of the N-heterocyclic carbene is from about 0.02% to about 40% by weight of the lipid material; the process is conducted at temperature from about 15° C. to about 75° C., at a pressure from about 40 kPa to about 1200 kPa, and for no longer than about 48 hours; and the conversion of glycerides to fatty acid alkyl esters is at least about 90%.

88. The process of claim 81, wherein the lipid material is food-based lipid waste, the alcohol is methanol; the concentration of the alcohol is from about 10% to about 40% by weight of the lipid material; the N-heterocyclic carbene is 1-butyl-3-methylimidzol-2-ylidene; the concentration of the N-heterocyclic carbene is from about 1% to about 20% by weight of the starting lipid material; the process is conducted at temperature of about 22° C., at a pressure of about 100 kPa, and for about 8 hours; and the conversion of glycerides to fatty acid alkyl esters is at least about 90%.
